(19) United States
(12) Patent Application Publication
(10) Pub. No.: US 2025/0090613 A1
(43) Pub. Date: Mar. 20, 2025

(54) AN ANTI-CANCER HERBAL COMPOSITION

(71) Applicants: Hareendran Nair J, Kerala (IN); Shan SASIDHARAN, Kerala (IN)

(72) Inventors: Hareendran Nair J, Kerala (IN); Shan SASIDHARAN, Kerala (IN)

(21) Appl. No.: 18/863,523

(22) PCT Filed: Feb. 20, 2023
(86) PCT No.: PCT/IN2023/050163

§ 371 Date: Nov. 6, 2024

(30) Foreign Application Priority Data
Feb. 21, 2022 (IN) 202141059578

(51) Int. Cl. A61K 36/48; A61K 9/48; A61K 31/355; A61P 35/00

(52) U.S. Cl. CPC A61K 36/48; A61K 9/4833; A61K 31/355; A61P 35/00; A61K 2236/35; A61K 2236/51

(57) ABSTRACT

The present invention relates to a disruptive therapeutic formula to manage prostate carcinoma from hexane extract of Caesalpinia bonduc seed by applying a novel ultrasonication assisted maceration and extraction technique. The present invention provides an anti-cancer herbal composition comprising: lyophilized, hexane extract of seeds of Caesalpinia bonduc, soyabean oil and primrose oil in 2:1 ratio respectively, tocopherol at a concentration of 100 mg/kg of the composition, wherein lyophilized hexane extract of seeds of Caesalpinia bonduc—soyabean oil and primrose oil is taken in 1:1 ratio respectively. The invention also relates to a method of extracting the active principals from the seeds of Caesalpinia bonduc.

### FIELD OF INVENTION

The invention relates to the field of herbal formulations. The invention specifically relates to a herbal formulation from the seeds of Caesalpinia bonduc for use in treatment of cancer.

### BACKGROUND OF INVENTION

Cancer is the second leading cause of death worldwide. Every year, approximately 10 million people are diagnosed with cancer, with half of them dying. Prostate cancer and breast cancer are the most common and lethal cancers in both men and women.

Medicinal plants are a better source of anti-cancer drugs than chemical drugs because of their low side effects. These plants are typically chosen based on ethnobotanical resources or traditional medicine. One of the most common methods for identifying new compounds used in chemotherapy and inhibiting the cancer process is screening plants and their phytochemicals as natural killer agents for cancer cells.

In 2018, prostate cancer was the second leading cause of cancer death in men, accounting for an estimated 1.28 million deaths. Based on clinical stage, histological grade and serum levels of prostate-specific antigen (PSA), current treatment options for prostate cancer include surgery, radiotherapy and/or chemotherapy. Such interventions are most effective in early disease, mainly if it is still localized to the prostate. Once the tumour has metastasized to other body organs, it becomes highly resistant to currently available treatment modalities. For prostate cancer patients with locally advanced or metastatic disease, androgen deprivation therapy is the standard of treatment as the pathogenesis of prostate cancer is highly dependent on androgen receptor signalling. However, the efficacy of androgen-blockade as a treatment for prostate cancer is limited. After an initial response to androgen deprivation therapy, most patients eventually progress to a highly aggressive, treatment-resistant form of the disease known as “castration-resistant” prostate cancer. This form of the disease poses a formidable therapeutic challenge and usually needs multiple combinations of therapeutic strategies to overcome. Treatment of prostate cancer is becoming increasingly intensive and aggressive, with a corresponding increase in resistance, toxicity and side effects. This has revived interest in non-toxic and cost-effective preventive strategies by utilizing traditional knowledge to develop novel formulations for managing prostate carcinoma.

Caesalpinia bonduc is one of the pantropical leguminous scandent shrubs that has been a very popular source of medicine since the ancient period by the local people and is being studied by several workers for their scientific use. Caesalpinia bonduc was ordered under the group of Caesalpiniaceae. It is otherwise called C. bonducella Flem. and C. crista Linn. Generally, it is called fever nut, bonduc nut and nicker nut also.

The pharmacological studies on C. bonduc seeds showed that the plant possessed anti-diarrhoeal, antiviral, antibacterial, antimicrobial, antifungal, antidiabetic, antitumor, antipyretic and analgesic, anti-filarial, anxiolytic, anti-inflammatory, antioxidant, immunomodulatory, and trypsin and chymotrypsin inhibitor properties.

The compounds reported from Caesalponia bonduc are phytosterols-sitosterol, heptocosane noncrystalline bitter glycoside bonducin, neutral saponin, noncrystalline bitter glycoside bonducin, terpenoids like caesalpin, β-caesalpin and α-caesalpin. Seed kernel were also rich in fatty oil, starch, sucrose, fatty acid-stearic, palmitic, oleic, linoceric, linolenic, and a mixture of unsaturated acids of low molecular weights, amino acid-aspartic acid, lysine, glycine, leucine, histidine, isoleucine, serine, r-amino-butyric acid, tyrosine, citrulline, glutamic acid, threonine, arginine, proline, L-alanine, methionine, phenyl alanine, cystine, valine, tryptophan.

Applicants found that the seed extract of Caesalpinia bonduc have anti-cancer properties. Therefore, the present invention provides herbal formulation of seed extracts of Caesalpinia bonduc for use in cancer treatment.

### SUMMARY OF INVENTION

The invention disclosed herein describes a disruptive therapeutic formula to manage prostate carcinoma from Caesalpinia bonduc seed (hexane extract) by applying a novel ultrasonication-assisted maceration and extraction technique. For this, healthy and disease-free seeds were selected. Following this, the seeds were subjected to various pharmacognostical analyses for authentication. Consequently, the seeds were crushed and subjected to a maceration process followed by ultrasonication to yield hexane extract. This process was repeated three times for an effective extract. After that, the marc was separated by filtration and then subjected to condensation using the soxhlet apparatus. After the completion of extraction, the hexane was completely evaporated using a rotary evaporator followed by lyophilization. This was again subjected to refinement using pure hexane to avoid unwanted substances. This was followed by LC-QTOF-MS/MS analysis to check the presence of various bioactive molecules. In addition to this, hexane extract also recorded significant cytotoxic activity against the L6 cell lines. Then the hexane extract was subjected to drug formulation by mixing the 50% of the product obtained after lyophilization was solubilized with 50% of a 2:1 ratio of pure refined soyabean oil and evening primrose oil, along with tocopherol as a preservative. Finally, this was subjected to soft gel capsule making using a soft gel capsule making apparatus.

One object of the invention is to provide a herbal formulation comprising seed extracts of Caesalpinia bonduc for use in treatment of cancer.

Another object of the invention is to provide a method of extracting active components from the seed of Caesalpinia bonduc for preparing a herbal formulation, for use in treatment of cancer.

In an embodiment the invention discloses an anti-cancer herbal composition comprising:
lyophilized, hexane extract of seeds of Caesalpinia bonduc,
soyabean oil and primrose oil in 2:1 ratio respectively,
tocopherol at a concentration of 100 mg/kg of the composition,
wherein lyophilized hexane extract of seeds of Caesalpinia bonduc—soyabean oil and primrose oil is taken in 1:1 ratio respectively.

In another embodiment the invention discloses a method of preparing lyophilized, hexane extract of seeds of Caesalpinia bonduc, wherein the method comprises the steps of:
a) collecting healthy germinating seed of Caesalpinia bonduc,
b) washing the seeds in water to remove impurities,
c) drying the seeds in an oven at a temperature of 50-75° C. for 30 to 90 minutes,
d) crushing the seeds into a colloidal mass,
e) macerating the seeds in 100% hexane solution,
f) taking the macerated seeds into a container and adding hexane to the container until the seeds are completely immersed in hexane solution,
g) providing intermittent shaking to the solution for about 24 hours,
h) subjecting the solution to sonication for 70-120 min at 45-55° C.,
i) providing intermittent shaking to the solution for about 24 hours,
j) repeating the process of steps h) and i) for two to three more times,
k) decant, filter and extract the marc,
l) evaporate the solution at a temperature of 50 to 60° C.,
m) condense the evaporated solution using soxhlet extraction process,
n) lyophilize the extract from soxhlet extraction process to get a powder.

In yet another embodiment the seed extract comprises (+)-α-tocopherol, fucosterol, linoleic acid, caesalpinia C, caesalpinin N, caesalmin E, caesalmin B, norcaesalpinin D, norcaesalpinin E, beta-Amyrin, campesterol, caesaldekarin H, caesaldekarin J, caesaldekarin K.

In another embodiment the herbal composition disclosed herein is made into soft gel capsules.

In a different embodiment the herbal composition disclosed herein is used in treatment of cancer.

### BRIEF DESCRIPTION OF DRAWINGS

The foregoing summary, as well as the following detailed description of the invention will be better understood when read in conjunction with the appended drawings. For the purpose of assisting in the explanation of the invention, there are shown in the drawings, embodiments which are presently preferred and considered illustrative. In the drawings:

FIG. 1 illustrates TLC profile of the seed extract of C. bonduc;

FIG. 2 illustrates LC-QTOF-MS/MS chromatogram of the hexane extract;

FIG. 3 illustrates cytotoxic activity of hexane extract against L6 cell lines.

### DETAILED DESCRIPTION OF INVENTION

The present invention will now be described more fully herein after. For the purposes of the following detailed description, it is to be understood that the invention may assume various alternative variations and step sequences, except where expressly specified to the contrary. Thus, before describing the present invention in detail, it is to be understood that this invention is not limited to particularly exemplified systems or embodiments that may of course, vary. The use of examples anywhere in this specification including examples of any terms discussed herein is illustrative only, and in no way limits the scope and meaning of the invention or of any exemplified term. Likewise, the invention is not limited to various embodiments given in this specification.

As used herein, the singular forms “a,” “an,” and “the” include plural reference unless the context clearly dictates otherwise. The term “and/or” means one or all of the listed elements or a combination of any two or more of the listed elements.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

When the term “about” is used in describing a value or an endpoint of a range, the disclosure should be understood to include both the specific value and endpoint referred to.

As used herein the terms “comprises”, “comprising”, “includes”, “including”, “containing”, “characterized by”, “having” or any other variation thereof, are intended to cover a non-exclusive inclusion.

The invention disclosed herein describes a disruptive therapeutic formula to manage prostate carcinoma from Caesalpinia bonduc seed (hexane extract) by applying a novel ultrasonication-assisted maceration and extraction technique. For this, healthy and disease-free seeds were selected. Following this, the seeds were subjected to various pharmacognostical analyses for authentication. Consequently, the seeds were crushed and subjected to a maceration process followed by ultrasonication to yield hexane extract. This process was repeated three times for an effective extract. After that, the marc was separated by filtration and then subjected to condensation using the soxhlet apparatus. After the completion of extraction, the hexane was completely evaporated using a rotary evaporator followed by lyophilization. This was again subjected to refinement using pure hexane to avoid unwanted substances. This was followed by LC-QTOF-MS/MS analysis to check for the presence of various bioactive molecules. In addition to this, hexane extract also recorded significant cytotoxic activity against the L6 cell lines. Then the hexane extract was subjected to drug formulation by mixing the 50% of the product obtained after lyophilization was solubilized with 50% of a 2:1 ratio of pure refined soyabean oil and evening primrose oil, along with tocopherol as a preservative. Finally, this was subjected to soft gel capsule making using a soft gel capsule making apparatus.

The best method of performing the invention will be described in detail with the following examples.

### Example 1

### Selection and Processing of Seed Samples for the Drug Formulation

Healthy and disease-free seeds were collected from the location coordinate 9.98°N 76.28° E (Ernakulam, Kerala) and the plants were cleaned under running tap water for 10 minutes to remove foreign matters and earthy materials. After that, it was rinsed twice with distilled water and air-dried to a constant weight at about 50-55° C. in an oven for about 1 h. After that, the seeds will be subjected to various pharmacognostical analyses for authentication of the sample before going for the formulation.

### Example 2

### Pharmacognostical Evaluation of the Seed

Organoleptic Evaluation of the Seed of C. bonduc

The organoleptic evaluation of the seed was conducted as per the standard procedures. The organoleptic description of the seed of C. bonduc is presented in Tables 1.

TABLE 1
Organoleptic evaluation of the seed of C. bonduc

| Parameters | Organoleptic properties |
|---|---|
| Color | Light brown |
| Odour | Characteristic |
| Taste | Astringent/bitter |
| Texture | Outer hard and inner soft |

Physicochemical Properties of C. bonduc

In addition to the organoleptic evaluation, we also conducted the physicochemical analysis of the seeds. Standard procedures were adopted for the process. The result of the phytochemical analysis of the C. bonduc seeds is presented in Table 2. The physicochemical analysis of C. bonduc powdered seeds reveals the parameters such as moisture content, total ash values, acid insoluble ash values, water-soluble ash values, alcohol soluble extractive values and water-soluble extractive values (Table 2). In addition to this, the physicochemical analysis also includes the extractive values in different solvents. Among the different solvents used, ethanol extract recorded the maximum extractive value of about 11.28%, followed by hexane, methanol and water.

TABLE 2
Physicochemical properties of C. bonduc

| Parameter | Results |
|---|---|
| Foreign matter (% w/w) | Nil |
| LOD (% w/w) | 6.61 |
| Ash values | |
| Total ash (% w/w) | 3.26 |
| Acid insoluble ash (% w/w) | 0.39 |
| Water soluble | 1.69 |
| Alcohol soluble extract (%) | 13.05 |
| Water soluble extract %) | 16.70 |
| Extractive values (%) | |
| Hexane | 4.9 |
| Toluene | 0.936 |
| Pet ether (40:60 + 60:80; 1:1) | 1.228 |
| Ethyl acetate | 1.04 |
| Chloroform | 0.496 |
| Acetone | 0.480 |
| Ethanol | 11.28 |
| Methanol | 3.224 |
| Water | 3.68 |
| Heavy metals (mg/kg) | |
| Arsenic | 0.14 |
| Cadmium | BDL |
| Lead | 0.08 |
| Mercury | 0.11 |

BDL—Below Detectable Level

### TLC Profile

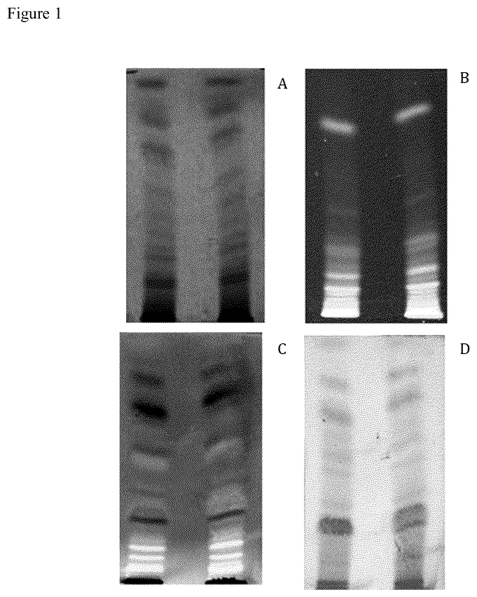

TLC profile of the seed extract were conducted. After developing the TLC profile, the spots were detected in short (254 nm) and long (366 nm) UV. At 254 nm, three major spots along with several minor spots were detected in the developed TLC of the raw seed extract with an Rf value range of 0.16-0.9. In the case of 366 nm, four spots were detected with an Rf value in the range of 0.12-0.89, whereas the derivatized sample recorded eight major spots with an Rf value in the range of 0.02-0.80. But the derivatized sample in visible light detected five major spots with an Rf value range of 0.05-0.80 (FIG. 1).

### Example 3

### Novel Ultrasonication Assisted Maceration/Extraction and Purification Process of the Hexane Extract

### Preparation of Seed for Solvent Extraction

The healthy, germinating seeds were selected for further processing. First, the selected seeds were subjected to heating at about 70° C. for about 1 h in a hot air oven. In the next step, seeds were crushed using an herb crusher or choppers to smash them into a colloidal mass. This process will reduce the size and increase the surface area to facilitate easier extraction of the active ingredients.

### Solvent Extraction Process

After crushing, the samples were treated with pure hexane (100%) and subjected to thorough maceration process.

### Ultrasonication Assisted Maceration

In this extraction procedure, coarsely powdered seeds were placed inside a container; the menstruum (hexane) was poured on top until it completely covered the crude drug material. The container was then closed and kept for about 24 h for maceration along with intermittent shaking. After that, this was subjected to sonication using an ultrasonicator (ultrasonication assisted maceration and extraction) for about 90 min at about 50° C. After that, it was again subjected to maceration for another 24 h with intermittent shaking. This was again subjected to ultrasonication for about 90 min at about 50° C. This process was repeated two more times for the complete extraction of the active drug material. The maceration process, followed by ultrasonication will be continued for three consecutive days or until complete extraction of the active components from the marc. This innovative ultrasonication process adopted here significantly improved the extraction of active ingredients from the seed. The content is stirred periodically, and if placed inside a bottle, it should be shaken from time to time to ensure complete extraction. At the end of extraction, the micelle is separated from the marc by filtration or decantation. Subsequently, the marc is then separated from the menstruum by evaporation in an oven or on top of a water bath at about 50-55° C.

### Soxhlet Extraction

After extraction, the marc was separated by filtration and subjected to condensation using the soxhlet apparatus. This process is also known as “continuous hot extraction.” The apparatus is called a “Soxhlet extractor” and is made of glass. It consists of a round bottom flask, an extraction chamber, a siphon tube, and a condenser at the top. The hexane used in the ultrasonication-assisted maceration/extraction process is poured into the bottom flask, followed by the thimble into the extraction chamber. This hexane was then heated from the bottom flask, evaporated, and passed through the condenser. It condenses and flows down to the extraction chamber extracting the drug by coming into contact. Consequently, when the level of hexane in the extraction chamber reaches the top of the siphon, the hexane and the extracted marc flow back to the flask. The entire process repeatedly continues until the marc is completely extracted, at which point the hexane flowing from the extraction chamber does not leave any residue behind.

After the completion of extraction, the hexane was completely evaporated using a rotary evaporator (100 rpm at about 35° C.) for the efficient and gentle removal of solvents from samples. This process gives a thick slurry. This was again subjected to refinement using pure hexane to avoid unwanted substances such as phosphatides, color-producing substances, residual soap or substances with an undesirable smell. Volatile compounds are removed by steam injection under a high vacuum during deodorization. After the refinement process, the extract was subjected to freeze-drying using a lyophilizer. The product thus obtained was used for the formulations.

### Example 4

### Detection of the Bioactive Compounds in the Extract Using LC/MS-QTOF Analysis

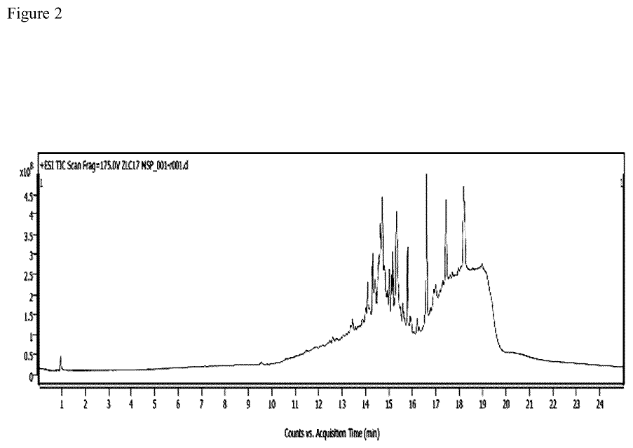

Qualitative tandem liquid chromatography quadrupole time of flight mass spectrometry (LC-QTOF-MS/MS) was utilized to identify compounds present in the extract. The data processing revealed the presence of 15 compounds in the extract from the seeds (FIG. 2 and Table 3).

TABLE 3
List of compounds detected in the hexane extract

| S, No | Name | Formula | RT | Mass |
|---|---|---|---|---|
| 1 | (+)-α-Tocopherol | C29 H50 O2 | 18.160 | 430.3805 |
| 2 | Fucosterol | C29 H48 O | 19.721 | 412.3682 |
| 3 | Linoleic acid | C18 H32 O2 | 15.800 | 280.2400 |
| 4 | Caesalpinin C | C24 H32 O6 | 15.501 | 416.2177 |
| 5 | Caesalpinin N | C23 H32 O6 | 14.189 | 404.2175 |
| 6 | Caesalmin E | C26 H36 O9 | 15.518 | 492.2335 |
| 7 | Caesalmin B | C22 H28 O6 | 14.189 | 388.1928 |
| 8 | Norcaesalpinin D | C25 H32 O9 | 14.421 | 476.2152 |
| 9 | Norcaesalpinin E | C21 H28 O6 | 14.887 | 376.1856 |
| 10 | Beta-Amyrin | C30 H50 O | 17.429 | 426.3819 |
| 11 | Campesterol | C28 H48 O | 18.658 | 400.3709 |
| 12 | Caesaldekarin H | C22 H32 O4 | 15.402 | 360.2273 |
| 14 | Caesaldekarin J | C21 H26 O4 | 12.378 | 342.1807 |
| 15 | Caesaldekarin K | C23 H34 O5 | 16.050 | 390.2420 |

### Example 5

### Novel Formula for Drug Formulation

The product obtained after lyophilization of the hexane extract was solubilized with 2:1 ratio of pure refined soyabean oil and evening primrose oil along with tocopherol as preservatives in 1:1 ratio. The concentration of tocopherol used here is 100 mg/kg. After that, it was subjected to soft gel capsule making using a soft gel capsule making machine (changsung softgel system). This soft gel capsule was used for various studies.

### Example 6

### Steps Involved in the Manufacturing of Soft Gel Capsule

### Material Selection and Dispensing

All raw materials are purchased from an established vendor.

### Gel Mass Preparation.

Dispensed materials are used for gel preparation.

The gel mass was prepared as per the product manufacturing process.

### Fill/Medicament Preparation (Hexane Extract).

Dispensed medicines are used for filling preparations.

Before dispensing the medicine (Hexane extract of C. bonduc), the following tests should be done:
The density of medicine
The viscosity of the medicine
The P^{h} and TDS of the medicine
LOD of the medicine
Microbiological testing (TPC (Total Plate Count) and TYMC (Total Yeast and Mold Count))

Fill was prepared in accordance with the product manufacturing process.

### Encapsulation and Semi Drying

Using an encapsulation machine, soft gel capsules are formed.

Using semi driers, the fresh soft gel capsules are partially dried so that they can be easily handled and spread on the trays.

### Tray Drying

Semi-dried capsules are spread on the trays and kept in the drying room at below 20% relative humidity till the moisture in the capsule shell is below 12% or the archived set hardness.

### Polishing

This process was performed before sorting and inspection to facilitate easy sorting and inspection.

During polishing, surface lubrication oil is removed from the capsules.
Also, polishing increases the quality and appearance of the capsules.

### Sorting

A sorting activity was performed on the polished capsules.

Sorting is done using the soft gel capsule sorting machine.

In this machine, different sized capsules are separated, like oversize, undersize, and good capsules.

### Production of a Gelatin Mass

Distribute a batch of raw materials
Transfer the dispensed materials to the shell manufacturing area.
Raise the temperature of the gel manufacturing mixer to 70-80° C.
Weigh purified water with glycerin, sorbitol and preservatives in a clean vessel. Load the above mixer by means of vacuum. Slowly combine and heat to 70-80° C.
Add gelatin in a uniform stream into above solution without forming lumps and continue cooking till the gelatin completely melts. Start de-aerating the mixture by applying vacuum.
Mix the opacifying and colouring agents into the glycerin water solution before adding it to the molten gel mentioned above. Mix well.
Unload into a pre heated gel holding tank.
Hold the gel for a minimum of 4 hrs at 50 to 60° C. for maturation before taking it for encapsulation.
Physical Check-Uniform mixing, free from particles and air bubble.

### Encapsulation Process

The encapsulation process is described as a form/fill/seal process.
Two flat ribbons of shell material are manufactured on the encapsulation machine and brought together on a twin set of rotating dies.
The dies contain recesses in the desired size and shape, which cut out the ribbons into two dimensional shapes and form a seal around the outside.
At the same time, a pump delivers a precise dose of fill material through a nozzle incorporated into a filling wedge whose tip sits between the two ribbons in between two die pockets at the point of cut out.
The wedge is heated to facilitate the sealing process.
The wedge injection causes the two flat ribbons to expand into the die pockets, giving rise to the three-dimensional finished product.
After encapsulation, the soft gels are dried for two days to two weeks, depending on the product.
Productivity increases or diminishes upon considering the following variables:—
   Density, Viscosity
   Capsule Size
   Capsule shape
   Quality of gel mass
   Machine Speed
   Experience of the operator

### Drying

Soft gel capsules are dried in two stages—

### Tumble Drying:—

Dry, dehumidify air is forced across the tumble and removes the moisture from the outer surface of the capsules.

### Supplemental Drying in the Drying Room:—

After the tumble dryers, the soft capsules are placed on special trays for final drying in the drying room for up to 48 hours or more, or until moisture in the shell is less than 12% or the capsules reach the desired hardness.

### Polishing

The capsules are polished in a conventional coating pan using lint free oil absorbent wipes to remove lubricant oil from the outer surface of the capsules. This also increases the appearance of the capsules.

### Sorting

Polished capsules are sorted to separate the good capsules from the overfilled and underfilled capsules using a sorting machine.

### Inspection

Soft gel capsules are inspected in 3 stages—

### Tray Dried Capsules

After the drying period completes, they are inspected on the individual trays for leakers, slugs, any other visible rejects.

### Final Inspection

After tray inspection, polishing and sorting, capsules are inspected either in an inspection machine or manually to remove physically defective capsules to make them ready for packing.

### Example 7

### Cytotoxicity Testing of the Hexane Extract in L6 Cell Lines

The MTT (3-(4, 5-dimethyl thiazol-2-yl)-2, 5-diphenyl tetrazolium bromide) assay was used to determine the anticancer activity of the hexane extract against L6 cell lines.

The MTT assay relies on the ability of viable cells' mitochondrial dehydrogenase enzyme to cleave the tetrazolium rings of the pale yellow MTT and form dark blue formazan crystals that are largely impermeable to cell membranes, resulting in its accumulation within healthy cells. The crystals are liberated as a result of the cells being dissolved with detergent. The number of surviving cells is proportional to the amount of formazan product produced. A simple colorimetric assay using a multi-well scanning spectrophotometer can then be used to quantify the colour (ELISA reader).

Cells (5×103/well) were seeded in 0.2 ml of medium (DMEM with 10% PBS) in 96 well plates, treated with drugs for 72 hours, and cytotoxicity was measured after incubation. Following the removal of the drug-containing media, 25 l of MTT solution (5 mg/ml in PBS) and 75 l of complete medium were added to the wells (untreated and treated) and incubated for 2 hours. At the end of the incubation, MTT lysis buffer (0.1 ml/well) was added to the wells and incubated for another 4 hours at 37° C. The optical densities at 570 nm were measured using a plate reader at the end of the incubation period. The relative cell viability in percentage (A 570 of the treated sample/A 570 of the untreated sample×100) was calculated.

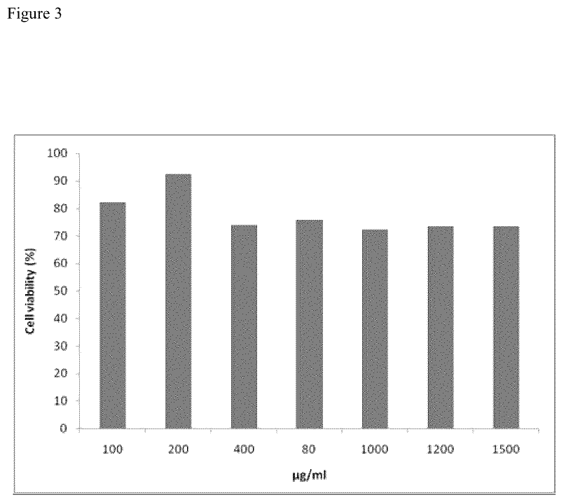

Hexane extract recorded significant cytotoxic activity is shown in FIG. 3.

The embodiments disclosed herein can be expressed in different forms and should not be considered as limited to the listed embodiments in the disclosed invention. The various embodiments outlined in the above sections are construed such that it provides a complete and a thorough understanding of the disclosed invention, by clearly describing the scope of the invention, for those skilled in the art.

We claim:

1. An anti-cancer herbal composition comprising:
lyophilized, hexane extract of seeds of Caesalpinia bonduc,
soyabean oil and primrose oil in 2:1 ratio respectively,
tocopherol at a concentration of 100 mg/kg of the composition,
wherein lyophilized hexane extract of seeds of Caesalpinia bonduc—soyabean oil and primrose oil is taken in 1:1 ratio respectively.

2. The herbal composition as claimed in claim 1 wherein the seed extract comprises (+)-α-tocopherol, fucosterol, linoleic acid, caesalpinia C, caesalpinin N, caesalmin E, caesalmin B, norcaesalpinin D, norcaesalpinin E, beta-Amyrin, campesterol, caesaldekarin H, caesaldekarin J, caesaldekarin K.

3. The herbal composition as claimed in claim 1 wherein the composition is made into soft gel capsules.

4. The herbal composition as claimed in claim 1 wherein the composition is used in treatment of cancer.

5. A method of preparing lyophilized, hexane extract of seeds of Caesalpinia bonduc claimed in claim 1, wherein the method comprises the steps of:
a) collecting healthy germinating seed of Caesalpinia bonduc,
b) washing the seeds in water to remove impurities,
c) drying the seeds in an oven at a temperature of 50-75° C. for 30 to 90 minutes,
d) crushing the seeds into a colloidal mass,
e) macerating the seeds in 100% hexane solution,
f) taking the macerated seeds into a container and adding hexane to the container until the seeds are completely immersed in hexane solution,
g) providing intermittent shaking to the solution for about 24 hours,
h) subjecting the solution to sonication for 70-120 min at 45-55° C.,
i) providing intermittent shaking to the solution for about 24 hours,
j) repeating the process of steps h) and i) for two to three more times,
k) decant, filter and extract the marc,
l) evaporate the solution at a temperature of 55 to 65° C.,
m) condense the evaporated solution using soxhlet extraction process,
n) lyophilize the extract from soxhlet extraction process to get a powder.

6. The method as claimed in claim 5 wherein the maceration can be carried out using ultrasonicator.
